(19) United States
(12) Patent Application Publication
(10) Pub. No.: US 2015/0313446 A1
(43) Pub. Date: Nov. 5, 2015

(54) ROBOTIC-ASSISTED SURGICAL SYSTEM AND CONTROL METHOD THEREOF

(71) Applicant: OLYMPUS CORPORATION, Tokyo (JP)

(72) Inventors: Ryohei OGAWA, Tokyo (JP); Kosuke KISHI, Tokyo (JP)

(73) Assignee: OLYMPUS CORPORATION, Tokyo (JP)

(21) Appl. No.: 14/795,205

(22) Filed: Jul. 9, 2015

Related U.S. Application Data
(63) Continuation of application No. PCT/JP2014/052595, filed on Jan. 29, 2014.
(60) Provisional application No. 61/760,718, filed on Feb. 5, 2013.

(51) Int. Cl. A61B 1/00; A61B 19/00; A61B 1/005

(52) U.S. Cl. CPC A61B 1/0016; A61B 1/005; A61B 19/2203; A61B 1/00006; A61B 1/00055; A61B 1/00045; A61B 19/30; A61B 19/46; A61B 1/00009; A61B 2019/2211; A61B 2019/302; A61B 2019/465

(57) ABSTRACT

A robotic-assisted surgical system includes a flexible inserted portion having an elongated shaft; a distal-end movement-amount detecting portion that detects an amount of movement of the distal end of the inserted portion; an operating unit that is disposed outside the body and that is operated by an operator outside the body; a driving portion that drives the inserted portion at a proximal end thereof in accordance with an operation signal input to the operating unit; and a control portion that calculates a difference between the amounts of movement of the distal end and the proximal end of the inserted portion, and that, in the case in which the difference is greater than a predetermined threshold, controls the driving portion so as to notifies the operator to that effect.

### CROSS-REFERENCE TO RELATED APPLICATIONS

This is a continuation of International Application PCT/JP2014/052595, with an international filing date of Jan. 29, 2014, which is hereby incorporated by reference herein in its entirety. This application claims the benefit of U.S. Provisional Patent Application No. 61/760,718, the content of which is incorporated herein by reference.

### TECHNICAL FIELD

The present invention relates to a robotic-assisted surgical system and a control method thereof.

### BACKGROUND ART

In the related art, there is a known electrically-driven bending endoscope in which a distal-end portion of an inserted portion is bent by pulling it with a wire by means of an electrically-powered motor, and external force exerted on the distal end of the inserted portion is estimated based on the amount by which the wire is displaced and tension detected by a tension sensor, the result of which is presented to an operator (for example, see Patent Literatures 1 and 2).

With such an electrically-driven bending endoscope, the operator inserts the inserted portion by means of electrical power while checking an endoscope image displayed on a monitor, until an affected portion appears in the endoscope image.

### CITATION LIST

### Patent Literature

{PTL 1} Publication of Japanese Patent No. 3549434
{PTL 2} Japanese Unexamined Patent Application, Publication No. 2010-35768

### SUMMARY OF INVENTION

An first aspect of the present invention is a robotic-assisted surgical system including a flexible inserted portion that has an elongated shaft adapted to be inserted into a body, and that is provided, at a distal end thereof, with an image observation system, which acquires an image of a body interior; a distal-end movement-amount detecting portion that detects an amount of movement of the distal end of the inserted portion; an operating unit that is disposed outside the body and that is operated by an operator; a driving portion that drives the inserted portion at a proximal end thereof in accordance with an operation signal input to the operating unit; and a control portion that controls the driving portion, wherein the control portion calculates a difference between the amount of movement of the distal end of the inserted portion, detected by the distal-end movement-amount detecting portion, and an amount of movement of the proximal end of the inserted portion upon being moved by the driving portion, and, in the case in which the difference is greater than a predetermined threshold, notifies the operator to that effect.

A second aspect of the present invention is a robotic-assisted surgical system including a flexible inserted portion that has an elongated shaft adapted to be inserted into a body, and that is provided, at a distal end thereof, with an image observation system, which acquires an image of a body interior; a distal-end movement-amount detecting portion that detects an amount of movement of the distal end of the inserted portion; a force detecting portion that is provided at the distal end of the inserted portion and that detects a contact pressure exerted on the internal tissue; an operating unit that is disposed outside the body and that is operated by an operator; a driving portion that drives the inserted portion at a proximal end thereof in accordance with an operation signal input to the operating unit; and a control portion that controls the driving portion, wherein the control portion calculates a difference between the amount of movement of the distal end of the inserted portion, detected by the distal-end movement-amount detecting portion, and an amount of movement of the proximal end of the inserted portion upon being moved by the driving portion, and, in the case in which the difference is greater than a predetermined movement-amount threshold or in the case in which the contact pressure detected by the force detecting portion is greater than a predetermined force threshold, notifies the operator to that effect.

A third aspect of the present invention is a control method of a robotic-assisted surgical system including driving a long, thin flexible inserted portion that is inserted into a body and that acquires an image of a body interior at a proximal end thereof; detecting an amount of movement of a distal end of the inserted portion; calculating a difference between the detected amount of movement of the distal end of the inserted portion and an amount of movement of the proximal end of the inserted portion; and controlling driving of the inserted portion at the proximal end thereof so as to, in the case in which the difference is greater than a predetermined threshold, notify the operator to the effect.

### BRIEF DESCRIPTION OF DRAWINGS

FIG. 1 is a diagram showing the overall configuration of a robotic-assisted surgical system according to an embodiment of the present invention.

FIG. 2 is a block diagram showing the robotic-assisted surgical system in FIG. 1.

FIG. 3 is a diagram showing an endoscope, which is a component of the robotic-assisted surgical system in FIG. 1, (a) in a state in which an inserted portion thereof is inserted into the large intestine, (b) in a state in which the inserted portion is advancing normally in the large intestine, and (c) in a state in which the inserted portion is abnormally bent in the large intestine.

FIG. 4 is a flowchart for explaining the operation of the robotic-assisted surgical system in FIG. 1.

FIG. 5 is a diagram showing a state in which an inserted portion of a first modification of the robotic-assisted surgical system in FIG. 1 is inserted into the large intestine.

FIG. 6 is a diagram showing a state in which an inserted portion of a second modification of the robotic-assisted surgical system in FIG. 1 is inserted into the large intestine.

FIG. 7 is a flowchart for explaining the operation of a third modification of the robotic-assisted surgical system in FIG. 1.

FIG. 8 is a perspective view showing a fourth modification of the robotic-assisted surgical system in FIG. 1, in a state in which the distal end of an inserted portion thereof is covered with a transparent cap.

FIG. 9 is a flowchart for explaining the operation of the robotic-assisted surgical system in FIG. 8.

FIG. 10 is a diagram showing an endoscope, which is a component of the robotic-assisted surgical system in FIG. 1, in a state in which the inserted portion thereof is inserted into the large intestine via an overtube.

### DESCRIPTION OF EMBODIMENT

A robotic-assisted surgical system 1 and a control method thereof according to an embodiment of the present invention will be described below with reference to the drawings.

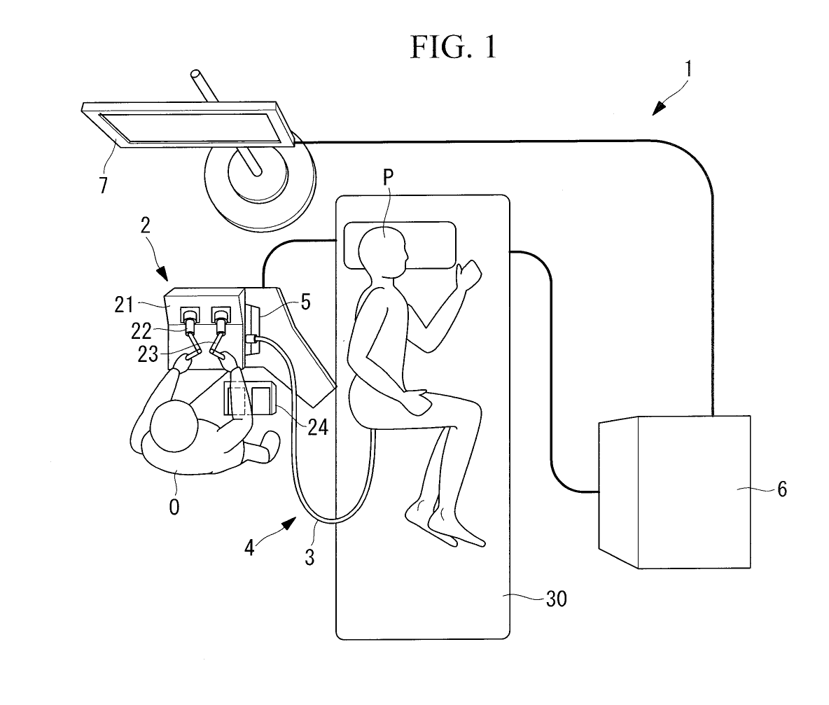

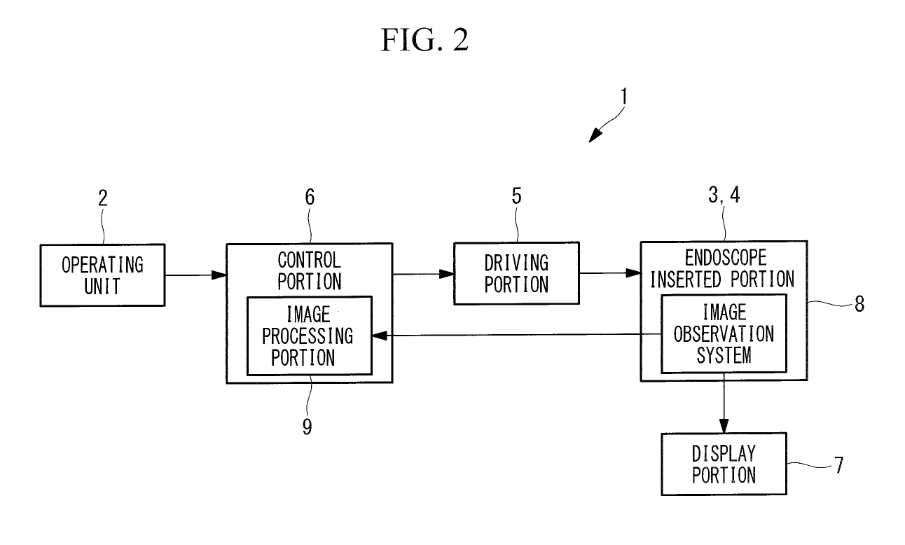

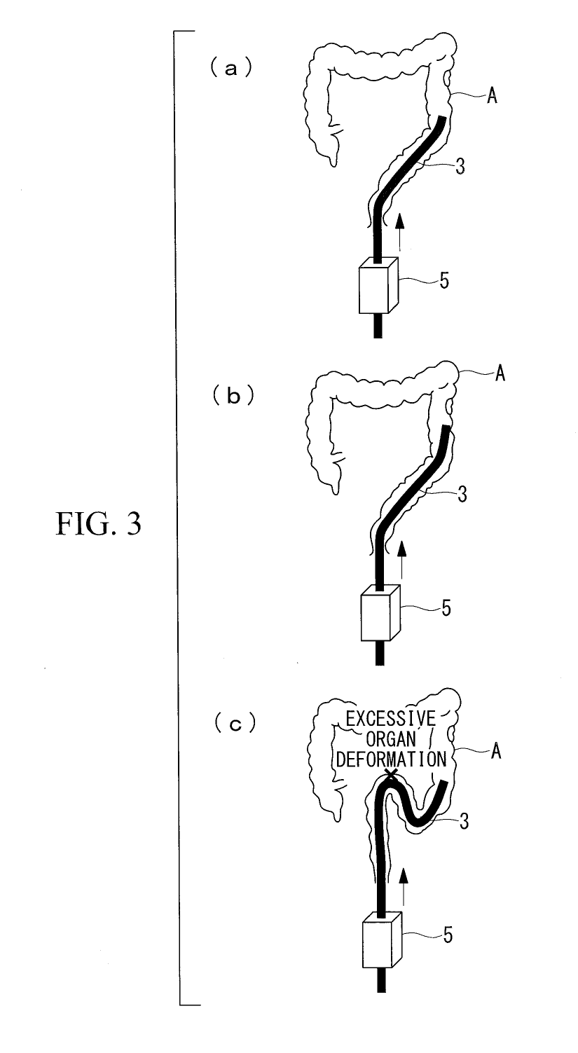

As shown in FIGS. 1 and 2, the robotic-assisted surgical system 1 according to this embodiment is an endoscope system employing a master-slave system, and is provided with an operating unit 2 that is operated by an operator O; an endoscope 4 having a flexible inserted portion 3 that is inserted into the body of a patient, for example, a soft organ A (see FIG. 3) such as the large intestine or the like; a driving portion 5 that drives the inserted portion 3 at the proximal end thereof to achieve inserting movement of the inserted portion 3 of the endoscope 4, bending movement at the distal end of the inserted portion 3, twisting movement of the inserted portion 3, and so forth; a control portion 6 that controls the driving portion 5; and a display portion 7 that displays an image acquired by the endoscope 4.

As shown in FIG. 1, the operating unit 2 has a pair of operation arms 22 and 23 attached to the operation table 21 and a footswitch 24 disposed on the floor.

The operation arms 22 and 23 have a multi-joint structure. The operation arm 22 is for performing bending operation of a bending portion of the inserted portion 3, and the operation arm 23 is for performing bending operation of a manipulator (not shown) provided at the distal end of the endoscope 4.

As shown in FIG. 1, an assistant (not shown) lays a patient P down on a surgical table 30 disposed closer to the operating unit 2 and performs appropriate procedures such as sterilization, anesthesia, and so forth.

The operator O instructs the assistant to introduce the inserted portion 3 into the large intestine from the anus of the patient P. The operator O operates the operation arm 22 to appropriately bend the bending portion of the inserted portion 3.

As shown in FIG. 2, at the distal end of the elongated shaft of the inserted portion 3, the endoscope 4 is provided with an image observation system 8 for acquiring an image of the body interior. The image acquired by the image observation system 8 is transmitted to an image processing portion 9 disposed in the control portion 6.

In the example shown in FIG. 3(a), the driving portion 5 is provided with an actuator for propelling the inserted portion 3 in the longitudinal direction. When the operator performs an operation for propelling the inserted portion 3 in the longitudinal direction at the operating unit 2, the actuator is driven and the inserted portion 3 is moved forward or backward in the longitudinal direction.

The control portion 6 generates command signals for the driving portion 5 to drive the actuator based on operation signals from the operating unit 2. Specifically, the control portion 6 calculates the amount of movement achieved, in a certain amount of time, by the proximal end of the inserted portion 3 due to the actuator and outputs command signals with which this amount of movement can be achieved to the driving portion 5.

In addition, upon receiving the image acquired by the image observation system 8, the control portion 6 identifies feature portions in the image by processing the image at the image processing portion 9, calculates the amount of movement of these feature portions in the certain amount of time, which is the same as the amount of time described above, and calculates the amount of movement of the distal end of the inserted portion 3 in the certain amount of time based on the amount of movement of these feature portions.

In addition, the control portion 6 calculates a difference between the calculated amount of movement of the proximal end of the inserted portion 3 due to the driving portion 5 and the amount of movement of the distal end of the inserted portion 3 calculated by processing the image, and compares the difference with a threshold.

Then, in the case in which the calculated difference is greater than the threshold, the control portion 6 restricts driving of the inserted portion 3 by the driving portion 5. Specifically, in the case in which the difference is greater than the threshold, the control portion 6 stops driving by the driving portion 5 in the direction that makes the inserted portion 3 advance, regardless of the operation signals input from the operating unit 2.

A control method of the thus-configured robotic-assisted surgical system 1 according to this embodiment will be described below with reference to the drawings.

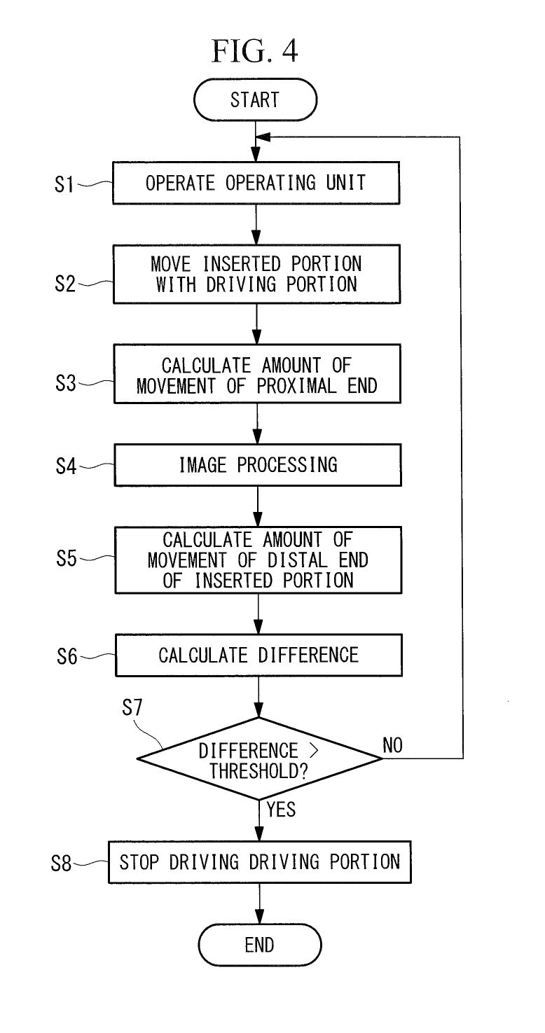

In order to perform observation and treatment inside the soft organ A, such as the large intestine, by employing the robotic-assisted surgical system 1 according to this embodiment, as shown in FIG. 4, the inserted portion 3 of the endoscope 4 is disposed in a state in which it is inserted into the organ A, and the operator operates the operating unit 2 (Step S1).

When the operating unit 2 is operated, the operation signals are output to the control portion 6 from the operating unit 2, and the command signals for moving the inserted portion 3 by the actuator of the driving portion 5 are generated at the control portion 6. By doing so, the actuator of the driving portion 5 is driven, thus moving the inserted portion 3 in accordance with the command signals (Step S2). When generating the command signals, the control portion 6 calculates the amount of movement achieved, in the certain amount of time, by the proximal end of the inserted portion 3 in accordance with the command signals (Step S3).

In addition, the control portion 6 receives the image transmitted thereto from the image observation system 8 of the inserted portion 3 and performs image processing thereof at the image processing portion 9 (Step S4).

The image processing portion 9 identifies the feature portions in the image and calculates the amount of movement of the feature portions in the certain amount of time, which is the same as the amount of time described above for which the amount of movement of the proximal end is calculated, by generating, for example, an optical flow. An optical flow indicates, by means of vectors, directions in which the plurality of feature portions have moved between two or more images acquired at time intervals.

Then, the control portion 6 calculates the amount of movement of the distal end of the inserted portion 3 in the longitudinal direction as a value proportional to the sum of the vectors constituting the optical flow generated by the image processing portion 9 or the variance thereof (Step S5).

In addition, the control portion 6 calculates the difference between the amount of movement of the proximal end of the inserted portion 3 and the amount of movement of the distal end thereof, which are calculated as described above (Step S6), and judges whether or not the calculated difference is greater than the threshold (Step S7).

When the difference is equal to or less than the threshold, the operating state can be judged to be normal, where the distal end is moving so as to follow the movement at the proximal end of the inserted portion 3, as shown in FIG. 3(b), and the processes from Step S1 are repeated.

On the other hand, when the difference is greater than the threshold, the distal end is not moving regardless of the movement at the proximal end of the inserted portion 3, for example, as shown in FIG. 3(c), and thus, the inserted portion 3 can be judged to be in the process of moving into an abnormal operating state in the organ A. Therefore, the control portion 6 stops the driving by the driving portion 5 in the direction that makes the inserted portion 3 advance, regardless of the operation signals input from the operating unit 2 (Step S8).

By doing so, it is possible to effectively prevent the problem of the organ A becoming deformed due to an increase in the degree of abnormality in the operating state of the inserted portion 3 in the organ A.

As described above, the robotic-assisted surgical system 1 and the control method thereof according to this embodiment afford an advantage in that, because the amount of movement of the distal end of the inserted portion 3 is calculated by applying image processing to the image acquired by the image observation system 8 of the endoscope 4, it is not necessary to provide any other special sensor, and it is possible to detect an abnormal operating state of the inserted portion 3 in the organ A in a simple manner.

Note that, in this embodiment, although the case in which the inserted portion 3 is moved in the longitudinal direction has been described as an example, the abnormal operating state may also be detected in a similar manner in the case in which the bending portion of the distal end of the inserted portion 3 is bent and the case in which the inserted portion 3 is made to perform twisting movement about the longitudinal axis thereof.

In the case when the bending portion is bent, because the generated optical flow will indicate parallel vectors directed in one direction, the average value thereof can be used as the amount of movement of the distal end.

In addition, in the case when the inserted portion 3 is twisted, because the generated optical flow will indicate spiraling vectors, vorticity or circulation is determined for the individual vectors by performing surface integration thereof, and the amount of movement of the distal end can be calculated as a value proportional thereto.

In addition, in the case in which the distal end is rotated despite a movement in the longitudinal axial direction, the distinguishing between the movement in the longitudinal axial direction and the rotation can be performed by comparing the vorticities or calculating the sum of vectors by focusing only on half of a screen. The case in which circulation of a vortex is greater than a predetermined value can be distinguished, as rotation, from the case in which circulation of a vortex is less than the predetermined value, which indicates a movement in the longitudinal axial direction. In addition, the case in which the sum of vectors in half of the screen is zero can be distinguished, as a movement in the longitudinal direction, from the case in which the sum of vectors in half of the screen is not zero, which indicates a rotational movement.

In addition, as the feature portions to be identified when performing image processing, although it suffices to use edges in an image, it is also effective to identify, as the feature portions, portions having characteristic shapes, such as a tumor, the tubular structure or the folded (haustra) structure of the large intestine A. In this case, image processing should be set to preferentially identify the above-described characteristic shapes.

In addition, it is also effective to identify portions having a characteristic color as the feature portions. For example, by setting the color of a local injection or the like in advance, portions having that color can preferentially be identified as the feature portions when performing image processing.

In addition, with this embodiment, although the forward movement of the inserted portion 3 in the longitudinal direction caused by the driving portion 5 is stopped when the difference between the amounts of movement of the distal end and the proximal end of the inserted portion 3 becomes greater than the threshold, alternatively, the speed of movement in that direction may be decreased. In addition, instead of restricting the movement caused by the driving portion 5 or in addition thereto, a notifying portion that notifies the operator that the threshold is exceeded may be provided.

As the notifying portion, it is possible to employ an arbitrary method, whereby the notification is issued by means of audio, light, vibration, or displaying something on the display portion 7.

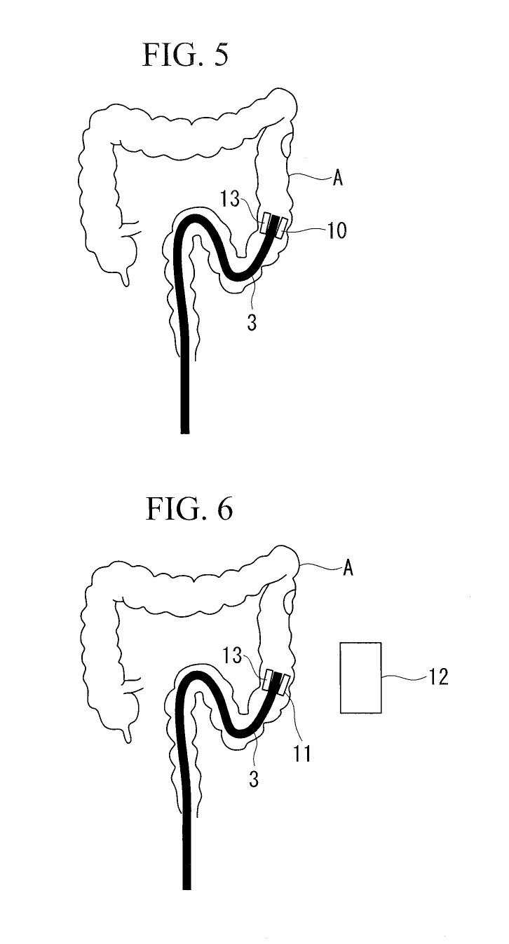

In addition, with this embodiment, although the amount of movement of the distal end of the inserted portion 3 is calculated by processing an image of the body interior acquired by the image observation system 8 of the endoscope 4, alternatively, as shown in FIG. 5, a sensor 10, such as an acceleration sensor, a gyro sensor, or an infrared distance sensor, that can calculate the amount of movement of the distal end of the inserted portion 3 based on a detection result may be provided at the distal end of the inserted portion 3.

In addition, as shown in FIG. 6, a detection target 11, such as a magnetic object or the like, may be fixed to the distal end of the inserted portion 3, and an amount of movement of the detection target 11 may be directly measured by means of a magnetometer (external sensor) 12 disposed outside the body. Note that the positions of the magnetometer 12 and the magnetic object 11 may be exchanged with each other. An X-ray opaque body may be employed instead of the magnetic object 11, and an X-ray imaging device may be employed instead of the magnetometer 12.

In addition, with this embodiment, although the movement of the inserted portion 3 caused by the driving portion 5 is always restricted when the difference between the amount of movement of the distal end of the inserted portion 3 and the amount of movement of the proximal end thereof exceeds the predetermined threshold, alternatively, in the above-described situation, it is also possible to switch between a mode in which the movement of the inserted portion 3 is restricted and a mode in which such a restriction is not applied. By doing so, an advantage is afforded in that, by using the non-restrictive mode in the case in which it is clear that the organ A will not be deformed, the operability can be enhanced.

In addition, with this embodiment, in addition to detecting the amount of movement of the distal end of the inserted portion 3, by disposing a force sensor 13 at the distal end of the inserted portion 3, the control portion 6 may restrict the operation of the driving portion 5 based on either the case in which the difference in the amounts of movement becomes greater than the threshold or the case in which contact pressure detected by the force sensor 13 becomes greater than a threshold. By doing so, it is possible to more reliably make the operator recognize an abnormal state of the inserted portion 3 in the body, even in the case in which one of the detection methods is not satisfactorily performed.

In addition, the control portion 6 may change the restriction on the operation of the driving portion 5 in a step-wise manner between the case in which the difference in the amounts of movement has become greater than the threshold and the case in which the contact pressure detected by the force sensor 13 has become greater than the threshold. Alternatively, the control portion 6 may restrict the operation of the driving portion 5 only in the case in which the difference in the amounts of movement has become greater than the threshold and the contact pressure detected by the force sensor 13 has also become greater than the threshold.

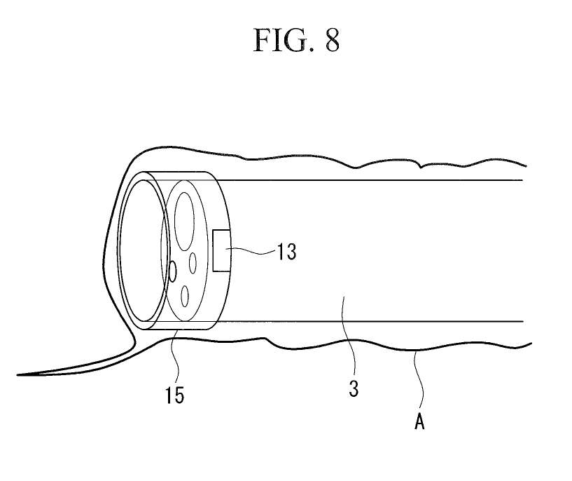

In addition, for example, when performing observation or the like of the large intestine, in some cases, the distal end of the endoscope 4 is covered with a transparent cap 15 and the inserted portion 3 is inserted while pressing this cap 15 against an inner wall of the large intestine (see FIG. 8). In this case, although the amount of movement of the distal end is decreased regardless of the amount of movement of the proximal end of the inserted portion 3, thus increasing the difference therebetween, it would be problematic if the operation of the driving portion 5 were restricted because of this.

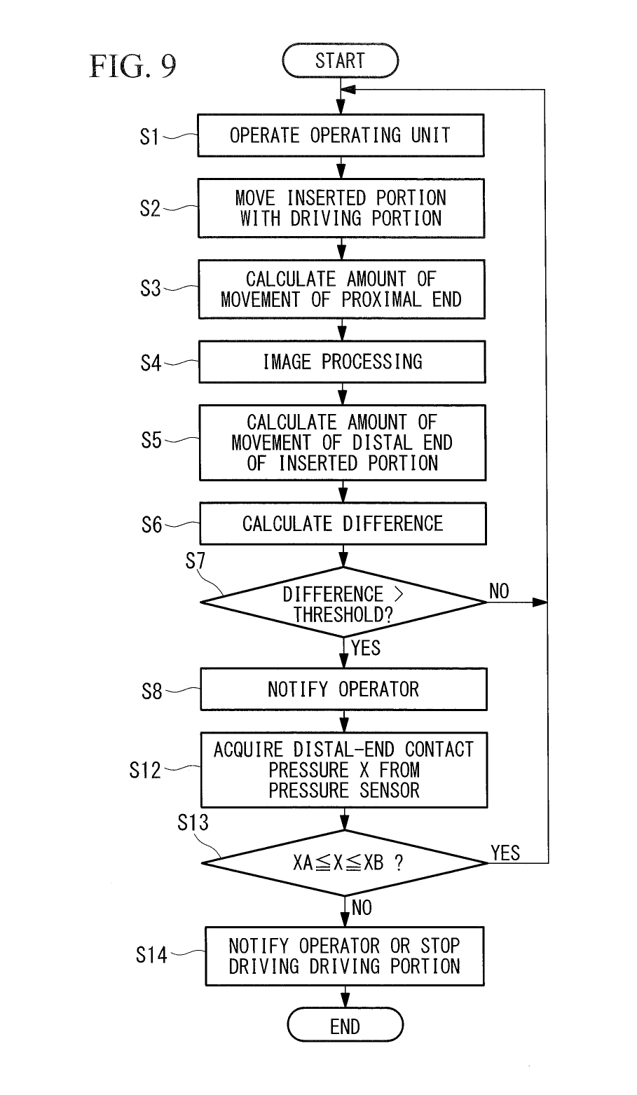

Therefore, two levels of thresholds (a threshold XA and a threshold XB, where XA<XB) may be provided to make a judgment regarding the contact pressure detected by the force sensor 13. The control method for the robotic-assisted surgical system 1 according to this embodiment employed in this case will be described below with reference to FIG. 9, in which processes up to Step S8 are the same as those described above.

Thus, in the case in which the difference between the amount of movement of the proximal end and the amount of movement of the distal end of the inserted portion is large, it is assumed that an abnormal state may possibly be occurring, and a notification is issued to the operator, and, subsequently, a distal-end contact pressure X is acquired from the force sensor 13 attached to the distal end (Step S12). In the case in which the contact pressure does not reach the smaller threshold XA, as presumed in the case in which the transparent cap 15 is pressed against the inner wall during insertion, as described above, it is assumed that the force may possibly be exerted on the organ A in an intermediate pathway for the inserted portion 3 because a presumed amount of force is not exerted on the transparent cap 15 at the distal end, and a notification to that effect may be issued, or the operation of the driving portion 5 may be restricted.

In the case in which the contact pressure X detected by the force sensor 13 exceeds the threshold XB for the pressure that is allowed to be exerted on the organ A, it is assumed that the transparent cap 15 may possibly be excessively pressed against the organ, and a notification to that effect may be issued or the operation of the driving portion 5 may be restricted. Specifically, options may be set based on the contact pressure X at the distal end (Step S13), the case in which XA≦X≦XB may be assumed to be within a presumed range, thus confirming normal operation, and, in cases other than that, a notification may be issued to the operator or the driving of the driving portion may be stopped (Step S14).

In addition, although the amount of movement of the distal end of the inserted portion 3 in a normal state is calculated by processing the image acquired by the image observation system 8, in the case in which it is difficult to calculate the amount of movement by means of image processing because the image observation system 8 is operating in a close viewing field, for example, in the case in which the image observation system 8 comes close to or comes into contact with internal tissue, making the entire image red or the like, the image observation system 8 may be judged to be operating in a close viewing field, and the operation may be switched so as to restrict the operation of the driving portion 5 based on the contact pressure detected by the force sensor 13.

Also, in addition to calculating the amount of movement of the distal end of the inserted portion 3 by performing image processing, the amount of movement of the distal end of the inserted portion 3 may be calculated at the same time by using another sensor 10, and the control portion 6 may restrict the operation of the driving portion 5 when the difference between at least one of the amounts of movement and the amount of movement of the proximal end of the inserted portion 3 becomes greater than the threshold. In this way also, it is possible to more reliably make the operator recognize an abnormal state of the inserted portion 3 in the body, even in the case in which one of the detection methods is not satisfactorily performed.

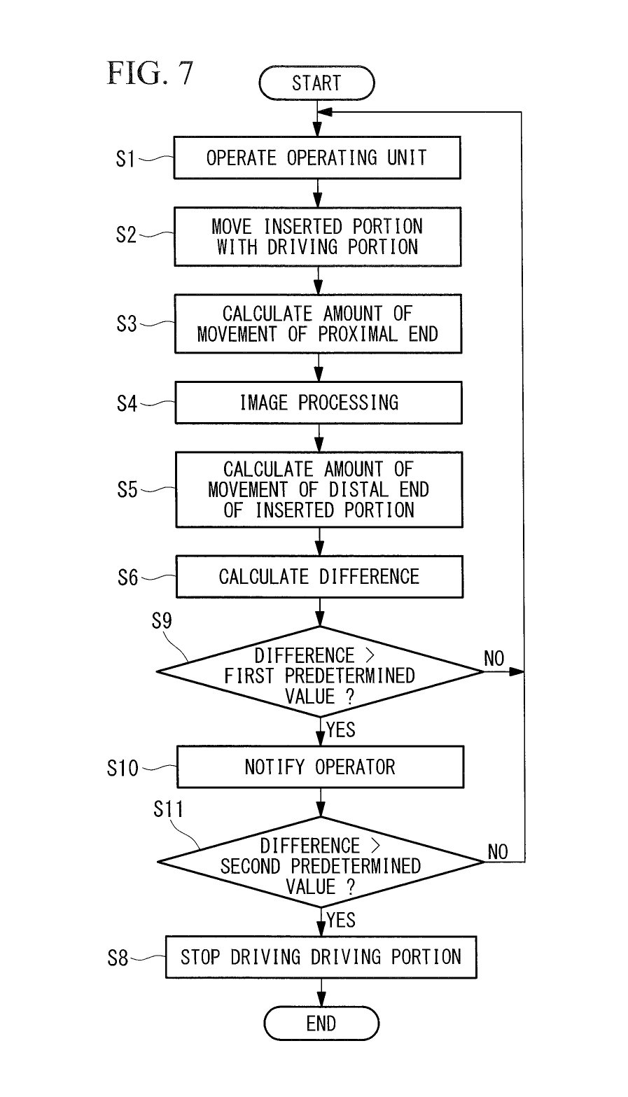

In addition, with this embodiment, although whether or not to restrict the operation of the driving portion 5 is determined based on one threshold, alternatively, this switching may be performed in a step-wise manner based on a plurality of thresholds. For example, as shown in FIG. 7, when the difference in the amounts of movement exceeds a first predetermined value (Step S9), this situation may be notified to the operator by displaying something or the like (Step S10), and, when the difference in the amounts of movement exceeds a second predetermined value that is greater than the first predetermined value (Step S11), the movement of the inserted portion 3 caused by the driving portion 5 may be restricted.

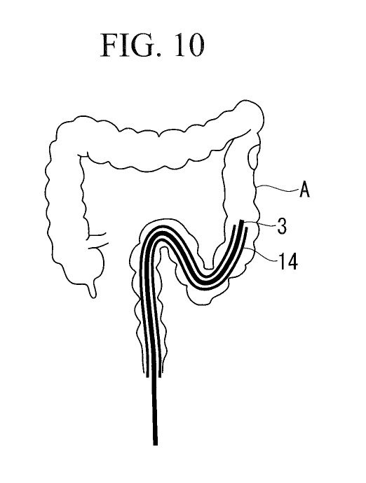

In addition, although cases in which the inserted portion 3 of the endoscope 4 is directly inserted into the organ A, such as the large intestine or the like, have been described in this embodiment, alternatively, as shown in FIG. 10, the present invention may be similarly applied to a case in which an overtube 14 is inserted into the organ A, and the inserted portion 3 of the endoscope 4 is inserted into the overtube 14. By doing so, the inserted portion 3 bends together with the overtube 14, and it is possible to prevent the organ A or the like from being subjected to an excessive burden.

In addition, with this embodiment, although the state of the inserted portion is judged based on a simple difference between the amount of movement at the distal end of the inserted portion 3 and the amount of movement at the proximal end of the inserted portion 3 in a certain amount of time, alternatively, the judgment may be made based on a difference between the amount of movement of the proximal end of the inserted portion 3 and a value obtained by multiplying the amount of movement of the distal end of the inserted portion, which is obtained by means of image processing, by a constant. The amount of movement of the distal end of the inserted portion 3 and the amount of movement of the proximal end thereof may be integrals for a certain amount of time or they may be integrals from the time at which driving was started.

In addition, the following aspects are lead from the individual embodiments described above.

An first aspect of the present invention is a robotic-assisted surgical system including a flexible inserted portion that has an elongated shaft adapted to be inserted into a body, and that is provided, at a distal end thereof, with an image observation system, which acquires an image of a body interior; a distal-end movement-amount detecting portion that detects an amount of movement of the distal end of the inserted portion; an operating unit that is disposed outside the body and that is operated by an operator; a driving portion that drives the inserted portion at a proximal end thereof in accordance with an operation signal input to the operating unit; and a control portion that controls the driving portion, wherein the control portion calculates a difference between the amount of movement of the distal end of the inserted portion, detected by the distal-end movement-amount detecting portion, and an amount of movement of the proximal end of the inserted portion upon being moved by the driving portion, and, in the case in which the difference is greater than a predetermined threshold, notifies the operator to that effect.

With this aspect, when the flexible inserted portion is inserted into the body and the operator operates the operating unit, the driving portion is driven based on the operation signal and the inserted portion is driven at the proximal end thereof. When the inserted portion is driven and the distal end is moved, the amount of movement of the distal end is detected by the distal-end movement-amount detecting portion. In this case, the difference between the detected amount of movement of the distal end and the amount of movement of the proximal end caused by the driving portion is calculated by the control portion, and, in the case in which the difference is greater than the predetermined threshold, the operator is notified to that effect. By doing so, the operator can confirm that the distal end is not being moved following the movement of the proximal end regardless of the amount of movement of the proximal end caused by the driving portion due to some abnormality occurring at the inserted portion, and thus, it is possible to restrict the inserted portion from being forcedly driven any further.

In the above-described first aspect, the distal-end movement-amount detecting portion may calculate the amount of movement of the distal end of the inserted portion by processing the image of the body interior acquired by the image observation system.

By doing so, the amount of movement of the distal end of the inserted portion can be calculated based on the image of the body interior acquired by the image observation system at the distal end of the inserted portion, and thus, an abnormality can be detected in a simple manner without requiring a separate sensor or the like. When calculating the amount of movement by means of image processing, an existing technique, such as point detection, edge detection, optical flow or the like, should be employed.

In the above-described first aspect, the distal-end movement-amount detecting portion may set a portion in the image having internal tissue with a characteristic shape as a feature portion, and may calculate the amount of movement of the distal end of the inserted portion based on an amount of movement of the feature portion.

By doing so, because the portion having the internal tissue with the characteristic shape can easily be identified in the image as the feature portion, the amount of movement of the distal end can be detected with high precision.

In the above-described first aspect, the distal-end movement-amount detecting portion may set a portion in the image having internal tissue of a characteristic color as a feature portion, and may calculate the amount of movement of the distal end of the inserted portion based on an amount of movement of the feature portion.

By doing so, because the portion having the internal tissue with the characteristic color can easily be identified in the image as the feature portion, the amount of movement of the distal end can be detected with high precision.

In the above-described first aspect, the distal-end movement-amount detecting portion may be provided with a sensor that is attached to the distal end of the inserted portion and that detects displacement, speed, or acceleration of the distal end of the inserted portion.

By doing so, the displacement, speed, or acceleration of the distal end of the inserted portion is detected by the operation of the sensor, and thus, the amount of movement of the distal end can directly be detected.

In the above-described first aspect, the distal-end movement-amount detecting portion may be provided with a detection target that is attached to the distal end of the inserted portion and an external sensor that is disposed outside the body and that detects a displacement of the detection target.

By doing so, the amount of movement of the detection target attached to the distal end of the inserted portion can directly be detected from outside the body by the external sensor.

In the above-described first aspect, the control portion may be provided with a notifying portion that, in the case in which the difference is greater than the predetermined threshold, notifies the operator to that effect by means of audio, a display, light, or vibration.

By doing so, the operator can reliably recognize an abnormality occurring at the inserted portion by means of audio, a display, light, or vibration generated by the notifying portion.

In the above-described first aspect, the control portion may control the driving portion so that, in the case in which the difference is greater than the predetermined threshold, the movement of the inserted portion is restricted.

By doing so, the control portion restricts the operation of the inserted portion by the driving portion, and thus, it is possible to reduce the burden on an organ or the like in the body caused by the operator continuing the operation further.

In the above-described first aspect, the control portion may control the driving portion so that, in the case in which the difference is greater than the predetermined threshold, the speed of the inserted portion is decreased.

By doing so, because the speed at which the inserted portion is moved by the driving portion is decreased by the control portion, it is possible to suppress an abrupt large deformation in an organ or the like in the body, even if the operator operates the operating unit when the inserted portion is in an abnormal state.

In the above-described first aspect, the control portion may activate the notifying portion in the case in which the difference is greater than a first predetermined value, and may control the driving portion so that, in the case in which the difference is greater than a second predetermined value that is greater than the first predetermined value, the movement of the inserted portion is restricted.

By doing so, it is possible to notify the operator about an abnormal state of the inserted portion in the body in a step-wise manner.

The above-described first aspect may be provided with a force detecting portion that is provided at the distal end of the inserted portion and that detects a contact pressure exerted on the internal tissue, wherein, in the case in which the difference between the amount of movement of the distal end of the inserted portion detected by the distal-end movement-amount detecting portion and the amount of movement of the proximal end of the inserted portion upon being moved by the driving portion is greater than the predetermined threshold, the control portion notifies the operator to that effect, and also controls the driving portion so that the movement of the inserted portion is restricted in the case in which the contact pressure detected by the force detecting portion falls outside of a presumed pressure range defined by two thresholds set in advance.

A second aspect of the present invention is a robotic-assisted surgical system including a flexible inserted portion that has an elongated shaft adapted to be inserted into a body, and that is provided, at a distal end thereof, with an image observation system, which acquires an image of a body interior; a distal-end movement-amount detecting portion that detects an amount of movement of the distal end of the inserted portion; a force detecting portion that is provided at the distal end of the inserted portion and that detects a contact pressure exerted on the internal tissue; an operating unit that is disposed outside the body and that is operated by an operator; a driving portion that drives the inserted portion at a proximal end thereof in accordance with an operation signal input to the operating unit; and a control portion that controls the driving portion, wherein the control portion calculates a difference between the amount of movement of the distal end of the inserted portion, detected by the distal-end movement-amount detecting portion, and an amount of movement of the proximal end of the inserted portion upon being moved by the driving portion, and, in the case in which the difference is greater than a predetermined movement-amount threshold or in the case in which the contact pressure detected by the force detecting portion is greater than a predetermined force threshold, notifies the operator to that effect.

With this aspect, the operator is notified not only in the case in which the amount of movement of the distal end of the inserted portion detected by the distal-end movement-amount detecting portion is extremely small with respect to the amount of movement of the proximal end thereof, but also in the case in which the contact pressure exerted on the internal tissue, which is detected by the force detecting portion provided at the distal end of the inserted portion, is greater than the force threshold, and therefore, the operator can more reliably recognize an abnormal state of the inserted portion in the body.

In the above-described second aspect, the distal-end movement-amount detecting portion may calculate the amount of movement of the distal end of the inserted portion by processing the image of the body interior acquired by the image observation system.

In the above-described second aspect, the control portion may judge whether or not the image observation system is operating in a close viewing field with respect to the internal tissue by processing the image, and, in the case in which a judgment indicating a close viewing field is obtained, may notify the operator based on the contact pressure detected by the force detecting portion.

By doing so, in the case in which the image observation system is operating in a close viewing field with respect to the internal tissue, although it becomes difficult to calculate the amount of movement of the distal end of the inserted portion by means of image processing, by detecting the contact pressure, it is possible to allow the operator to recognize an abnormal state of the inserted portion in the body.

A third aspect of the present invention is a control method of a robotic-assisted surgical system including driving a long, thin flexible inserted portion that is inserted into a body and that acquires an image of a body interior at a proximal end thereof; detecting an amount of movement of a distal end of the inserted portion; calculating a difference between the detected amount of movement of the distal end of the inserted portion and an amount of movement of the proximal end of the inserted portion; and controlling driving of the inserted portion at the proximal end thereof so as to, in the case in which the difference is greater than a predetermined threshold, notify the operator to the effect.

In the above-described third aspect, the amount of movement of the distal end of the inserted portion may be calculated by processing the acquired image.

In addition, in the above-described third aspect, control may be performed so as to restrict the movement of the inserted portion in the case in which the difference is greater than the predetermined threshold.

### REFERENCE SIGNS LIST

1 robotic-assisted surgical system
2 operating unit
3 inserted portion
5 driving portion
6 control portion
7 display portion (notifying portion)
8 image observation system
9 image processing portion (distal-end movement-amount
detecting portion)
10 sensor
11 detection target
12 magnetometer (external sensor)
13 force sensor (force detecting portion)

1. A robotic-assisted surgical system comprising:
a flexible inserted portion that has an elongated shaft adapted to be inserted into a body, and that is provided, at a distal end thereof, with an image observation system, which acquires an image of a body interior;
a distal-end movement-amount detecting portion that detects an amount of movement of the distal end of the inserted portion;
an operating unit that is disposed outside the body and that is operated by an operator;
a driving portion that drives the inserted portion at a proximal end thereof in accordance with an operation signal input to the operating unit; and
a control portion that controls the driving portion,
wherein the control portion calculates a difference between the amount of movement of the distal end of the inserted portion, detected by the distal-end movement-amount detecting portion, and an amount of movement of the proximal end of the inserted portion upon being moved by the driving portion, and, in the case in which the difference is greater than a predetermined threshold, notifies the operator to that effect.

2. The robotic-assisted surgical system according to claim 1, wherein the distal-end movement-amount detecting portion calculates the amount of movement of the distal end of the inserted portion by processing the image of the body interior acquired by the image observation system.

3. The robotic-assisted surgical system according to claim 2, wherein the distal-end movement-amount detecting portion sets a portion in the image having internal tissue with a characteristic shape as a feature portion, and calculates the amount of movement of the distal end of the inserted portion based on an amount of movement of the feature portion.

4. The robotic-assisted surgical system according to claim 2, wherein the distal-end movement-amount detecting portion sets a portion in the image having internal tissue of a characteristic color as a feature portion, and calculates the amount of movement of the distal end of the inserted portion based on an amount of movement of the feature portion.

5. The robotic-assisted surgical system according to claim 1, wherein the distal-end movement-amount detecting portion is provided with a sensor that is attached to the distal end of the inserted portion and that detects displacement, speed, or acceleration of the distal end of the inserted portion.

6. The robotic-assisted surgical system according to claim 1, wherein the distal-end movement-amount detecting portion is provided with a detection target that is attached to the distal end of the inserted portion and an external sensor that is disposed outside the body and that detects a displacement of the detection target.

7. The robotic-assisted surgical system according to claim 1, wherein the control portion is provided with a notifying portion that, in the case in which the difference is greater than the predetermined threshold, notifies the operator to that effect by means of audio, a display, light, or vibration.

8. The robotic-assisted surgical system according to claim 1, wherein the control portion controls the driving portion so that, in the case in which the difference is greater than the predetermined threshold, the movement of the inserted portion is restricted.

9. The robotic-assisted surgical system according claim 8, wherein the control portion controls the driving portion so that, in the case in which the difference is greater than the predetermined threshold, the speed of the inserted portion is decreased.

10. The robotic-assisted surgical system according to claim 7, wherein the control portion activates the notifying portion in the case in which the difference is greater than a first predetermined value, and controls the driving portion so that, in the case in which the difference is greater than a second predetermined value that is greater than the first predetermined value, the movement of the inserted portion is restricted.

11. The robotic-assisted surgical system according to claim 1, further comprising:
a force detecting portion that is provided at the distal end of the inserted portion and that detects a contact pressure exerted on the internal tissue,
wherein, in the case in which the difference between the amount of movement of the distal end of the inserted portion detected by the distal-end movement-amount detecting portion and the amount of movement of the proximal end of the inserted portion upon being moved by the driving portion is greater than the predetermined threshold, the control portion notifies the operator to that effect, and also controls the driving portion so that the movement of the inserted portion is restricted in the case in which the contact pressure detected by the force detecting portion falls outside of a presumed pressure range defined by two thresholds set in advance.

12. A robotic-assisted surgical system comprising:
a flexible inserted portion that has an elongated shaft adapted to be inserted into a body, and that is provided, at a distal end thereof, with an image observation system, which acquires an image of a body interior;
a distal-end movement-amount detecting portion that detects an amount of movement of the distal end of the inserted portion;
a force detecting portion that is provided at the distal end of the inserted portion and that detects a contact pressure exerted on the internal tissue;
an operating unit that is disposed outside the body and that is operated by an operator;
a driving portion that drives the inserted portion at a proximal end thereof in accordance with an operation signal input to the operating unit; and
a control portion that controls the driving portion,
wherein the control portion calculates a difference between the amount of movement of the distal end of the inserted portion, detected by the distal-end movement-amount detecting portion, and an amount of movement of the proximal end of the inserted portion upon being moved by the driving portion, and, in the case in which the difference is greater than a predetermined movement-amount threshold or in the case in which the contact pressure detected by the force detecting portion is greater than a predetermined force threshold, notifies the operator to that effect.

13. The robotic-assisted surgical system according to claim 12, wherein the distal-end movement-amount detecting portion calculates the amount of movement of the distal end of the inserted portion by processing the image of the body interior acquired by the image observation system.

14. The robotic-assisted surgical system according to claim 13, wherein the control portion judges whether or not the image observation system is operating in a close viewing field with respect to the internal tissue by processing the image, and, in the case in which a judgment indicating a close viewing field is obtained, notifies the operator based on the contact pressure detected by the force detecting portion.

15. A control method of a robotic-assisted surgical system comprising:
driving a long, thin flexible inserted portion that is inserted into a body and that acquires an image of a body interior at a proximal end thereof;
detecting an amount of movement of a distal end of the inserted portion;
calculating a difference between the detected amount of movement of the distal end of the inserted portion and an amount of movement of the proximal end of the inserted portion; and
controlling driving of the inserted portion at the proximal end thereof so as to, in the case in which the difference is greater than a predetermined threshold, notify the operator to the effect.

16. The control method of a robotic-assisted surgical system according to claim 15, wherein the amount of movement of the distal end of the inserted portion is calculated by processing the acquired image.

17. The control method of a robotic-assisted surgical system according to claim 15, wherein control is performed so as to restrict the movement of the inserted portion in the case in which the difference is greater than the predetermined threshold.
